(19) United States
(12) Patent Application Publication
(10) Pub. No.: US 2021/0038190 A1
(43) Pub. Date: Feb. 11, 2021

(54) METHOD AND SYSTEM FOR IDENTIFYING A MATERIAL OF INTEREST

(71) Applicant: ENDRA Life Sciences Inc., Ann Arbor, MI (US)

(72) Inventor: Jang Hwan Cho, Ann Arbor, MI (US)

(73) Assignee: ENDRA Life Sciences Inc., Ann Arbor, MI (US)

(21) Appl. No.: 16/535,635

(22) Filed: Aug. 8, 2019

(51) Int. Cl. A61B 8/00; A61B 5/00; A61B 8/14; A61B 8/08

(52) U.S. Cl. CPC A61B 8/4416; A61B 5/0093; A61B 8/469; A61B 8/08; A61B 8/5207; A61B 8/5223; A61B 8/14

(57) ABSTRACT

A method for identifying a material of interest comprises directing, using a radio frequency (RF) applicator, one or more RF energy pulses into a region of interest, the region of interest comprising the material of interest and at least one reference that are separated by at least one boundary; detecting, using an acoustic receiver, at least one multi-polar acoustic signal generated in the region of interest in response to the RF energy pulses; processing the at least one multi-polar acoustic signal to determine an electric field strength at the boundary; and identifying the material of interest based at least on the determined electric field strength.

### FIELD

The subject disclosure relates to thermoacoustic imaging and in particular to a method and system for identifying a material of interest.

### BACKGROUND

Materials can be identified or quantified by making one or more measurements of the material and as a result, if the type of material is unknown, identification or quantification methods can be performed to identify the material. These methods often require the use of specialized equipment. For example, dielectric measurement systems require a specialized probe and network analyzer.

In medical settings such as in a hospital, this specialized equipment may not be readily available. As will be appreciate, if required, it is desirable to determine one or more parameters of a material of interest using equipment that is readily available in a medical setting.

Although techniques for determining one or more parameters of a material have been considered, improvements are desired. It is therefore an object at least to provide a novel method and system for identifying a material of interest.

### SUMMARY

It should be appreciated that this summary is provided to introduce a selection of concepts in a simplified form that are further described below in the detailed description. This summary is not intended to be used to limit the scope of the claimed subject matter.

Accordingly, in one aspect there is provided a method for identifying a material of interest, the method comprising: directing, using a radio frequency (RF) applicator, one or more RF energy pulses into a region of interest, the region of interest comprising the material of interest and at least one reference that are separated by at least one boundary; detecting, using an acoustic receiver, at least one multi-polar acoustic signal generated in the region of interest in response to the RF energy pulses; processing the at least one multi-polar acoustic signal to determine an electric field strength at the at least one boundary; and identifying the material of interest based at least on the determined electric field strength.

In one or more embodiments, the identifying comprises calculating a parameter as a product of a Grüneisen Parameter of the material of interest and a conductivity of the material of interest.

In one or more embodiments, the identifying comprises looking up the calculated parameter in a lookup table.

In one or more embodiments, the electric field strength is determined based on an input power of the RF applicator and an attenuation coefficient of the reference.

In one or more embodiments, the electric field strength is determined based on an estimated thickness of the reference and an attenuation coefficient of the reference.

In one or more embodiments each multi-polar acoustic signal corresponds to a separate boundary location.

In one or more embodiments, detecting the at least one multi-polar acoustic signal is achieved using a thermoacoustic imaging system.

In one or more embodiments, the method further comprises directing, using an ultrasound system, sound waves into the region of interest; detecting, using an ultrasonic transducer of the ultrasound system, echoes generated in the region of interest in response to the sound waves; and processing ultrasound data associated with the echoes to generate one or more or more ultrasound images.

In one or more embodiments, identifying the material of interest is further based on at least one physical characteristic of the material of interest.

In one or more embodiments, the at least one physical characteristic is at least one of color, transparency, odor, texture and material state.

In one or more embodiments, the material of interest is tissue within a human body and the reference is lean tissue within the human body.

According to another aspect there is provided a system for identifying a material of interest, the system comprising: a thermoacoustic imaging system comprising a radio frequency (RF) applicator configured to emit RF energy pulses into the region of interest comprising a material of interest and a reference separated by at least one boundary and an acoustic receiver configured to receive at least one multi-polar acoustic signal generated in the region of interest in response to the RF energy pulses; and one or more processors configured to process multi-polar acoustic signals received by the acoustic receiver to determine an electric field strength at the boundary; and identify the material of interest based at least on the determined electric field strength.

In one or more embodiments, during the identifying the one or more processors are configured to calculate a parameter as a product of a Grüneisen Parameter of the material of interest and a conductivity of the material of interest.

In one or more embodiments, during the identifying the one or more processors are configured to look up the calculated parameter in a lookup table.

In one or more embodiments, the electric field strength is determined based on an input power of the RF applicator and an attenuation coefficient of the reference.

In one or more embodiments, the electric field strength is determined based on an estimated thickness of the reference and an attenuation coefficient of the reference.

In one or more embodiments, each multi-polar acoustic signal corresponds to a separate boundary location.

In one or more embodiments, the one or more processors are configured to adjust a frequency of the RF applicator.

In one or more embodiments, the material of interest is tissue within a human body and the reference is lean tissue within the human body.

In one or more embodiments, the reference is made of a known material and is placed adjacent to the material of interest.

In one or more embodiments, the reference is one of a container and a pad.

According to another aspect there is provided a non-transitory computer readable medium having stored thereon computer program code executable by one or more processors to: process at least one multi-polar acoustic signal generated in a region of interest comprising a material of interest and at least one reference that are separated by at least one boundary to determine an electric field strength at the boundary; and identify the material of interest based at least on the determined electric field strength

In one or more embodiments, the computer program code is executable by the one or more processors to calculating a parameter as a product of a Grüneisen Parameter of the material of interest and a conductivity of the material of interest.

In one or more embodiments, the computer program code is executable by the one or more processors to identify the material of interest by looking up the calculated parameter in a lookup table.

### BRIEF DESCRIPTION OF THE DRAWINGS

Embodiments will now be described more fully with reference to the accompanying drawings in which:

FIG. 1 is a schematic view of an imaging system wherein a region of interest is in vitro;

FIG. 2 is a graph showing exemplary multi-polar signals generated in response to thermoacoustic imaging of a tissue region of interest comprising different tissue materials separated by a boundary;

FIG. 3 is a graph showing exemplary electric field strength attenuation curves;

FIG. 4 is a graph showing exemplary flux (energy gradient) of RF energy pulses;

FIG. 5 is a flowchart of a method for identifying a material of interest;

FIG. 6 is a flowchart of another method for identifying a material of interest;

FIG. 7 is a schematic view of the imaging system with a region of interest is in vivo; and

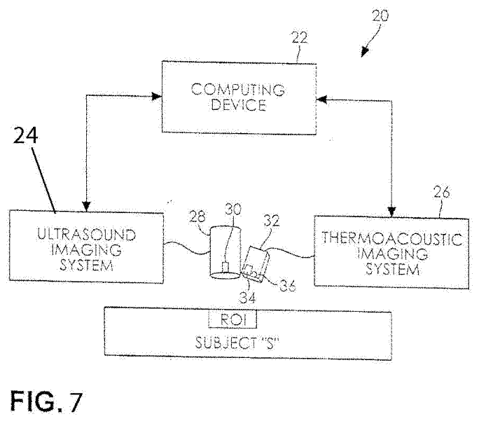

FIG. 8 is a schematic view of another embodiment of an imaging system.

### DETAILED DESCRIPTION OF THE EMBODIMENTS

The foregoing summary, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Moreover, unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientation depicted in the figures.

In the following, a method and system for identifying a material of interest are described. The method comprises directing, using a radio frequency (RF) applicator, one or more RF energy pulses into a region of interest, the region of interest comprising the material of interest and at least one reference that are separated by at least one boundary. Using an acoustic receiver, at least one multi-polar acoustic signal generated in the region of interest in response to the RF energy pulses is detected. The at least one multi-polar acoustic signal is processed to determine an electric field strength at the boundary. The material of interest is identified using at least the determined electric field strength.

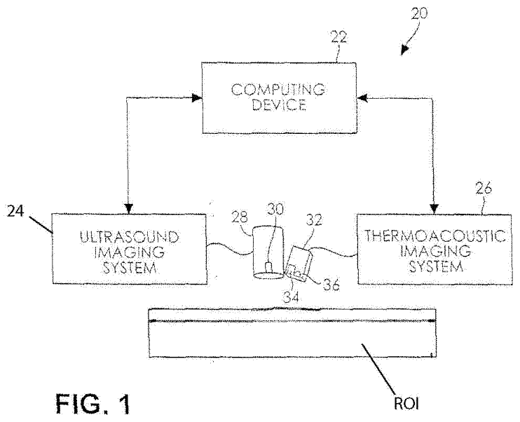

Turning now to FIG. 1, an exemplary imaging system is shown and is generally identified by reference numeral 20. As can be seen, the imaging system 20 comprises a programmed computing device 22 communicatively coupled to an ultrasound imaging system 24 and to a thermoacoustic imaging system 26. The ultrasound imaging system 24 and thermoacoustic imaging system 26 are configured to obtain ultrasound image data and thermoacoustic image data, respectively, of a region of interest ROI.

The programmed computing device 22 in this embodiment is a personal computer or other suitable processing device comprising, for example, a processing unit comprising one or more processors, system memory (volatile and/or non-volatile memory), other non-removable or removable memory (e.g., a hard disk drive, RAM, ROM, EEPROM, CD-ROM, DVD, flash memory, etc.) and a system bus coupling the various computer components to the processing unit. The computing device 22 may also comprise networking capabilities using Ethernet, Wi-Fi, and/or other suitable network format, to enable connection to shared or remote drives, one or more networked computers, or other networked devices. One or more input devices, such as a mouse and a keyboard (not shown) are coupled to the computing device 22 for receiving operator input. A display device (not shown), such as one or more computer screens or monitors, is coupled to the computing device 22 for displaying one or more generated images that are based on ultrasound image data received from the ultrasound imaging system 24 and/or the thermoacoustic image data received from thermoacoustic imaging system 26. The programmed computing device 22 executes program code stored on a computer readable medium and performs methods according to the program code as will be described in more detail below.

The ultrasound imaging system 24 comprises an acoustic receiver in the form of an ultrasound transducer 28 that houses one or more ultrasound transducer arrays 30 configured to emit sound waves into the region of interest ROI. Sound waves directed into the region of interest ROI echo off materials within the region of interest ROI, with different materials reflecting varying degrees of sound. Echoes that are received by the one or more ultrasound transducer arrays 30 of the ultrasound transducer 28 are processed by the ultrasound imaging system 24 before being communicated as ultrasound image data to the computing device 22 for further processing and for presentation on the display device as ultrasound images that can be interpreted by an operator. In this embodiment, the ultrasound imaging system 24 utilizes B-mode ultrasound imaging techniques assuming a nominal speed of sound of 1,540 m/s. As ultrasound imaging systems are known in the art, further specifics of the ultrasound imaging system 24 will not be described further herein.

The thermoacoustic imaging system 26 comprises an acoustic receiver in the form of a thermoacoustic transducer 32. The thermoacoustic transducer 32 houses one or more thermoacoustic transducer arrays 34 as well as a radio frequency (RF) applicator 36. It will however be appreciated that the RF applicator 36 may be housed separately from the thermoacoustic transducer 32. The RF applicator 36 is configured to emit short pulses of RF energy that are directed into materials within the region of interest ROI. In this embodiment, the RF applicator 36 has a frequency between about 10 Mhz and 100 GHz and has a pulse duration between about 0.1 nanoseconds and 10 nanoseconds. RF energy pulses delivered to materials within the region of interest ROI heat the materials thereby to induce acoustic pressure waves within the region of interest ROI that are detected by the thermoacoustic transducer 32. Acoustic pressure waves that are detected by the thermoacoustic transducer 32 are processed and communicated as thermoacoustic image data to the computing device 22 for further processing and for presentation on the display device as thermoacoustic images that can be interpreted by the operator.

In this embodiment, the ultrasound transducer 28 and thermoacoustic transducer 32 are mechanically interconnected so that the spatial relationship between the one or more ultrasound transducer arrays 30, the one or more thermoacoustic arrays 34 and the RF applicator 36 are known. The spatial relationship is set using a centerline of the one or more ultrasound transducer arrays 30, the one or more thermoacoustic transducer arrays 34, and RF applicator 36. The centerline of the ultrasound transducer array 34 and the thermoacoustic transducer array 34 is defined as being a mid-point of an area of the respective transduce array.

In this embodiment, the spatial relationship between the one or more ultrasound transducer arrays 30 and the one or more thermoacoustic transducer arrays 34 is such that the centerline of the one or more thermoacoustic transducer arrays 34 is set at a known angle a with respect to the centerline (also known as the axial axis or ultrasound transducer array beam axis) of the one or more ultrasound transducer arrays 30. The spatial relationship between the one or more thermoacoustic transducer arrays 34 and the RF applicator 36 is such that the centerline of the RF applicator 36 is spaced-apart and generally parallel to the centerline of the one or more thermoacoustic transducer arrays 34.

The imaging system 20 utilizes the known spatial relationship between the one or more ultrasound transducer arrays 30 and the one or more thermoacoustic transducer arrays 34 to increase the precision and accuracy of thermoacoustic imaging.

The coordinate system of the one or more ultrasound transducer arrays 30 of the ultrasound transducer 28 and the coordinate system of the one or more thermoacoustic transducer arrays 34 of the thermoacoustic transducer 32 are mapped by the computing device 22 so that acquired ultrasound and thermoacoustic images can be registered. Alternatively, the thermoacoustic imaging system 26 may make use of the one or more ultrasound transducer arrays 30 of the ultrasound transducer 28 by disconnecting the one or more ultrasound transducer arrays 30 from the ultrasound transducer 28 and connecting the one or more ultrasound transducer arrays 30 to the thermoacoustic transducer 32. As will be appreciated, by doing this coordinate mapping between the one or more ultrasound transducer arrays 28 and the one or more thermoacoustic transducer arrays 34 is not required.

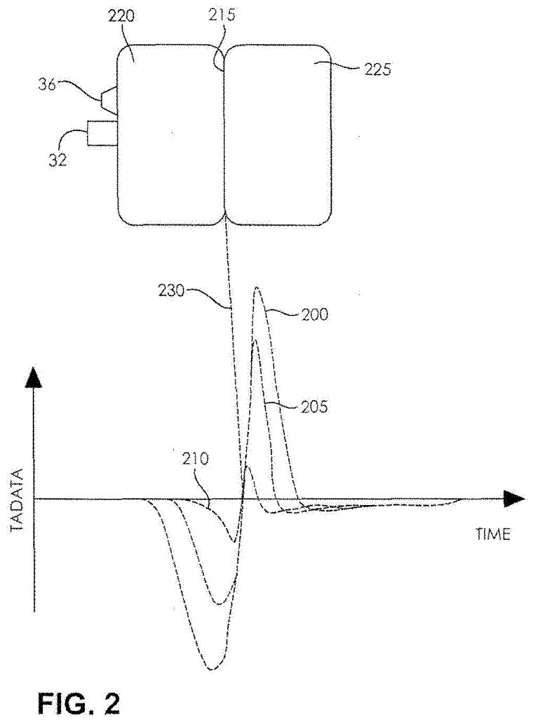

Exemplary multi-polar acoustic signals 200, 205 and 210 are shown in FIG. 2. The multi-polar acoustic signals 200, 205 and 210 are generated in response to thermoacoustic imaging of a tissue region of interest ROI comprising a first tissue 220 and a different type of second tissue 225 that are separated by a boundary 215. The dashed line 230 indicates a time point corresponding to the boundary 215. The differences in the peak-to-peak values of the multi-polar acoustic signals 200, 205 and 210 represent the extent to which the first tissue 220 expands into the boundary 215 and into the second tissue 225 before contracting. As the difference between the amount of energy absorbed of the two different tissues at the boundary 215 increases, the amount that the first tissue 220 expands into the boundary 215 and into the second tissue 225 increases. Therefore, the peak-to-peak amplitude of each multi-polar acoustic signal 200, 205 and 210 is proportional to the difference between the amount of energy absorbed of the two different tissues at the boundary 215. As can be seen, the peak-to-peak value of multi-polar acoustic signal 200 is greater than that of multi-polar acoustic signals 205, 210 and the peak-to-peak value of multi-polar acoustic signal 205 is greater than that of multi-polar acoustic signal 210. As such, the difference between the amount of energy absorbed of the two different tissues at the boundary 215 when multi-polar acoustic signal 200 is generated is greater than the difference between the amount of energy absorbed of the two different tissues at the boundary 215 when multi-polar signal 205 is generated. Similarly, the difference between the amount of energy absorbed of the two different tissues at the boundary 215 when multi-polar acoustic signal 205 is generated is greater than the difference between the amount of energy absorbed of the two different tissues at the boundary 215 when multi-polar signal 210 is generated.

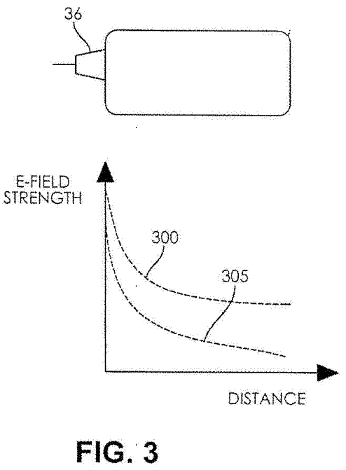

FIG. 3 shows electric field strength attenuation curves 300, 305 in material 310, 315 as a function of distance from the RF emitter 112 of a thermoacoustic imaging system.

The example is simplified and ignores factors such as reflections off an object boundary. Each electric field strength attenuation curve 300, 305 represents the electric field strength attenuation of material 310, 315, respectively, as a function of distance from the RF applicator 36. Each electric field strength attenuation curve 300, 305 corresponds to a material, each of which has a different attenuation coefficient (which could correspond to a different fat concentration for each respective material). The material associated with electric field strength curve 300 has a lower attenuation coefficient than the material associated with electric field strength curve 305. In one embodiment, the material with a lower attenuation coefficient is has a high fat concentration (e.g. greater than 10%) and the material with a higher attenuation coefficient has a low fat concentration (e.g. less than 10%).

The material 310 associated with electric field strength attenuation curve 300 has a different Grüneisen parameter than the material 315 associated with electric field strength attenuation curve 305.

Different materials (e.g. tissues) have characteristic dielectric properties at a given frequency and a temperature. The dielectric properties of a material determines how much energy is absorbed by the material. An electric field transmitted through the material is attenuated, and the amount of attenuation is determined by both dielectric and physical properties of the material. As an example, compared to normal tissue, fatty tissue absorbs less energy and thus attenuates less electric field. Knowing these properties, the amount of attenuation through a material can be estimated. Furthermore, for a given RF applicator with specific design and tuning, dielectric properties of a material lead to different RF matching and energy delivery. For example, if the applicator is tuned to match well on human body, it is likely to match poorly to material with high water content, such as ultrasound gel. Therefore, knowing the RF power and matching properties gives information on the material in contact with the applicator.

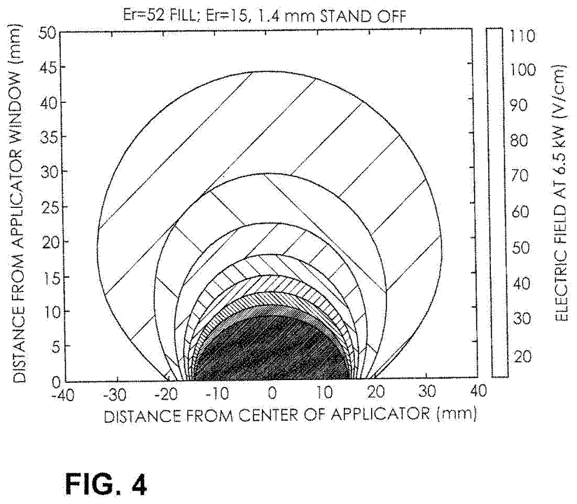

FIG. 4 shows the flux (energy gradient) of an RF energy pulse generated by the RF applicator 36. The RF applicator 36 is located and centered at the 0 value of the x-axis. As can be seen, as the distance from the center of the RF applicator 36 increases, the electric field strength decreases.

Different materials have characteristic dielectric properties at a given frequency. The dielectric properties of a material determine how much energy is absorbed thereby. An electric field transmitted through the material is attenuated, and the amount of attenuation is determined by both dielectric and physical properties of the material. As an example, compared to lean tissue, fatty tissue absorbs less energy and thus attenuates less electric field. Knowing these properties, the amount of attenuation can be estimated. Furthermore, for a given RF applicator with specific design and tuning, dielectric properties of a material lead to different RF matching and energy delivery. For example, if the RF applicator is tuned to match that of the human body, it is likely to match poorly to material with high water content, such as ultrasound gel. Therefore, knowing the RF power and matching properties give information on the material in contact with the RF applicator.

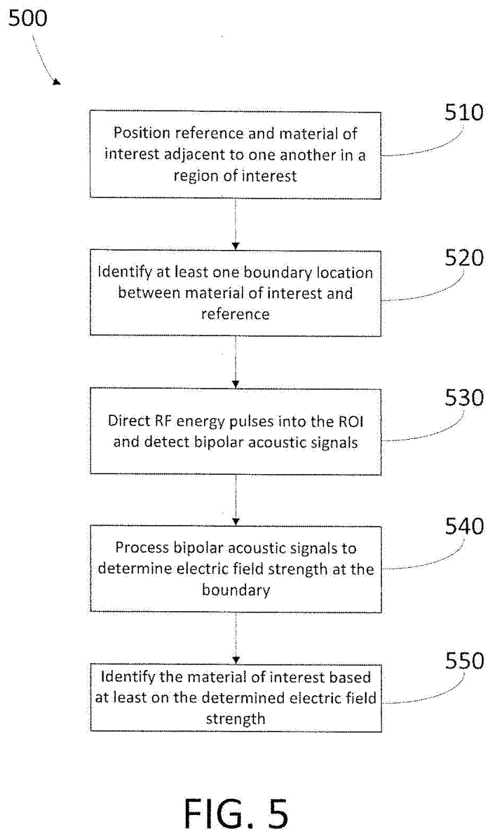

Turning now to FIG. 5, a method for identifying a material of interest is shown and is generally identified by reference numeral 500. Initially during the method, a reference and a material of interest are positioned adjacent to one another within a region of interest ROI (step 510).

In an embodiment that is outside of a human body, the reference can be in the form of a pad and is made of a known material, such as for example rubber. Other examples of pad construction can include but are not limited to gels made of materials such as agar, gelatin, gelwax, or the like. The pad should have known dielectric properties, low acoustic attenuation properties, minimal RF interference properties, and good acoustic matching properties. The reference or pad is over top of the material of interest.

At least one boundary location between the material of interest and the reference is identified in the reconstructed ultrasound image (step 520). At the boundary location, multi-polar acoustic signals are generated that are detected and received by the thermoacoustic transducer 32 (step 530).

The multi-polar acoustic signals received by thermoacoustic transducer 32 are communicated as thermoacoustic data to the computing device 22 for processing (step 540). In this embodiment, the computing device 22 is programmed to process the multi-polar acoustic signals to determine an electric field strength at the boundary.

The thermoacoustic pressure p(r, t) produced by a heat source H(r, t) obeys the following equation:

$${∇}^{2} p ( \underline{r} , t ) - \frac{1}{{c}^{2}} \frac{{\partial }^{2}}{\partial {t}^{2}} p ( \underline{r} , t ) = - \frac{β}{{C}_{p}} \frac{\partial }{\partial t} H ( \underline{r} , t ) \qquad ( 1 )$$

where r is the spatial position vector, β is the isobaric volume expansion coefficient, c is the sound speed and Cₚ is the specific heat capacity. Solving equation 1 with respect to the acoustic pressure wave p(r, t) yields the following forward problem:

$${p ( \underline{r} , t ) = \frac{β}{4 π {C}_{p}} \int \int \int \frac{\partial \underline{r}}{\vert \underline{r} - {\underline{r}}^{′} \vert} \frac{\partial H ( {\underline{r}}^{′} , {t}^{′} )}{\partial t ′} \vert}_{{t}^{′} = t - \frac{{\underline{r}}^{′}}{C}} \qquad ( 2 )$$

The heat source H(r, t) is modeled as the product of two factors, which are the spatial distribution of energy absorption A(r) and the temporal irradiation function l(t). The spatial distribution of energy absorption A(r) is determined based on characteristics of the materials(s) being imaged. Since the thermoacoustic transducer array 30 has a finite bandwidth, received thermoacoustic data p_{d}(r, t) is a result of the convolution of acoustic pressure wave p(r, t) and the impulse response of the thermoacoustic transducer array 30 h(t) as set out in equation 3:

p_{d}(r, t)=p(r, t)*ₜh(t)

where *ₜ denotes a one-dimensional temporal convolution.

As will be appreciated, for conventional thermoacoustic imaging, the goal is to recover the spatial absorption distribution A(r) by inverting the forward problem. As such, the irradiation function is modeled as a temporal function that is uniform at a given time point.

Due to the limited bandwidth of the thermoacoustic transducer array 30, accurately recovering the absorption distribution is not trivial. As such, extracting quantitative information requires sophisticated methods beyond that of conventional reconstruction methods.

When the material of interest is heated with a pulse of RF energy, the power deposition per unit volume A(r) is expressed as:

A(r)=ωε₀εᵣ″E²(r) (4)

where ω is the radian frequency, ε₀ is the vacuum permittivity, εᵣ″ is the imaginary part of the relative permittivity (also referred to as the conductivity) of the material of interest and ε(r) is the electric field strength. The strength of thermoacoustic data S(r) obtained from the material of interest is the product of the deposited energy and the Grüneisen parameter of the tissue F:

S(r)=γA(r)=γωε₀εᵣE²(r) (5)

Within a dielectric lossy medium, the electric field strength is attenuated as it propagates through the medium. The amount of attenuation is determined by various factors such as for example characteristics of region of interest and characteristics of the RF applicator 36. The spatial distribution of the electric field is:

E(r)=E₀E_{A}(r) (6)

where E₀ is the maximum electric field strength of the region of interest and E_{A}(r) is the attenuation of the electric field over a given space. For a simple 1D case, the attenuation E_{A}(r) can be expressed in exponential form:
E_{A}(d)=e^{-ηd}
where η is the electric field absorption coefficient of the region of interest and d is the distance of the region of interest from the RF applicator 36.

In this embodiment, equation 5 is used as a model to infer material parameters from the thermoacoustic data. As mentioned, thermoacoustic data obtained from the region of interest is in the form of multi-polar acoustic signals. The strength or peak-to-peak amplitudes of the multi-polar acoustic signals represent the absorption property difference between the material of interest and the reference. Further, the phase of the thermoacoustic data at the boundary indicates which material (the material of interest or the reference) has a higher or lower absorption coefficient. The strength or peak-to-peak amplitudes Sₗ of each multi-polar acoustic signal measured at the boundary location, r, is expressed in equation 8:

Sₗ=(Γ_{MOI}_{^{ε}}_{r,MOI}−Γ_{ref}_{^{ε}}_{r,ref})ωε₀Eₗ² (8)

where MOI denotes the material of interest, ref denotes the reference, and Eₗ denotes the incident electric field strength at the boundary.

As shown in equation 8, the strength of the multi-polar acoustic signal is determined by material parameters and the strength of the electric field at the boundary.

Since the properties of the reference are known, to estimate the properties of the material of interest, only the strength of the electric field at the boundary is required. Put another way, since the material of interest has different dielectric and/or thermoacoustic properties than the reference, the properties of the material of interest can be deduced.

Using equation 6, the incident electric field Eₗ at the boundary location (going from the reference to the material of interest) can be estimated as:

E=E₀e^{-η}^{_{ref}}^{d}^{_{ref}} (9)

where E₀ is the electric field strength at the start of the reference, η_{ref} is the attenuation coefficient of the reference, and d_{ref} is a thickness of the reference. As will be appreciated, the electric field strength E₀ may be modeled using a finite-difference time domain (FDTD method) or may be inferred from measurements taken at the RF applicator 36. The electric field strength E₀ may alternatively be directly measured at the boundary using an electric field probe.

The multi-polar acoustic signal strength at the boundary location can be derived from equations 8 and 9:

$$\begin{matrix}{S}_{l} = ( {Γ}_{MOI} {ɛ}_{r , MOI}^{″} - {Γ}_{ref} {ɛ}_{r , ref}^{″} ) {ωɛ}_{0} {E}_{l}^{2} \\ = ( {Γ}_{MOI} {ɛ}_{r , MOI}^{″} - {Γ}_{ref} {ɛ}_{r , ref}^{″} ) {{ωɛ}_{0} ( {E}_{0} {e}^{- {n}_{ref} {d}_{ref}} )}^{2}\end{matrix} \qquad ( 10 )$$

As such, parameter k_{MOI} of the material of interest can be calculated as:

$${k}_{MOI} = {Γ}_{MOI} {ɛ}_{r , MOI}^{″} = \frac{{s}_{l}}{{{ωɛ}_{0} ( {E}_{0} {e}^{- {n}_{ref} {d}_{ref}} )}^{2}} + {Γ}_{ref} {ɛ}_{0} {ɛ}_{r , ref}^{″} \qquad ( 11 )$$

The parameter k_{MOI} is a property of a material that is related to thermoacoustic signal generation (see equation 5). The thermoacoustic signal is determined by this parameter, E-field strength, and object independent parameters (such as frequency).

Once the parameter k_{MOI} is calculated, the material of interest can be identified (step 550). In this embodiment, the material of interest can be identified using a lookup table generated from previously performed models and experiments. Table 1 illustrates an exemplary lookup table:

TABLE 1
Grüneisen Parameter and Conductivity
of Materials at Room Temperature (at 434 MHz)

| | Distilled | Subcutaneous | | | Mineral |
|---|---|---|---|---|---|
| | Water | fat | Muscle | Blood | Oil |
| Grüneisen | 0.11 | 0.81 | 0.21 | 0.14 | 0.71 |
| parameter | | | | | |
| (Γ) | | | | | |
| Conductivity | 0.045 | 0.042 | 0.8 | 1.36 | 0 |
| (ε″ᵣ) (S/m) | | | | | |
| Parameter | 0.00495 | 0.03402 | 0.168 | 0.1904 | 0 |
| (k) | | | | | |

Those skilled in the art will appreciate that other parameters or properties may be used to help identify the material of interest. For example, a lookup table comprising physical characteristics of the material of interest may be used. Table 2 illustrates an exemplary lookup table:

TABLE 2
Physical Characteristics of Materials

| | Distilled | | Mineral | |
|---|---|---|---|---|
| | water | Fat | oil | Concrete |
| Color | colorless | white | colorless | white/gray/ |
| | | | | colored |
| Transparency | transparent | opaque | transparent | opaque |
| Odor | odorless | rancid | odorless | concrete odor |
| texture | smooth | smooth | smooth | rough |
| material state | liquid | solid | liquid | solid |

Those skilled in the art will appreciate that the ultrasound imaging system may be used to help identify the material of interest. For example, the speed of sound and scattering properties of the material of interest can be observed and compared to the speed of sound and scattering properties of the reference. The ultrasound imaging system may also be used to inspect a condition of the material of interest and to check for undesirable structures such as for example cracks or air bubbles that may interfere with the thermoacoustic imaging process.

Those skilled in the art will appreciate that radio frequency (RF) characteristics may be used to help identify the material of interest. For example, the RF forward and reflected power of the RF energy pulses may be monitored. As is known, the RF forward power is the power of the RF energy pulses emitted by the RF applicator. The RF reflected power is the power of the RF energy pulses that are reflected back to the RF applicator. Using the RF forward and reflected powers, the voltage standing wave ratio (VSWR) can be calculated and is a measure of how efficiently RF power is being transmitted from the RF applicator.

In this embodiment, RF energy pulses are directed through at least one intermediate area and into a region of interest that comprises the material of interest. The RF energy pulses have a known frequency and a known amplitude. A first power monitor is used to measure the forward power of the RF energy pulses. A second power monitor is used to measure the reflected power of the RF energy pulses. The RF forward and reflected powers may be acquired with or without the reference present.

In this embodiment, the VSWR is calculated as a ratio of the measured forward power and the measured reflected power. Using the measured forward power, the VSWR, and an estimated thickness of the at least one intermediate area, a parameter of the material of interest may be estimated using a lookup table.

Those skilled in the art will appreciate that the phase of the multi-polar acoustic signals may be used to help identify the material of interest. Since the reference is made of a known material, the material (the material of interest or the reference) that absorbs more heat than the other material expands rapidly across the boundary and into the other material, that expands less, and then quickly contract. The phase of the multi-polar acoustic signal depends on which one of the materials absorbs more heat.

Those skilled in the art will appreciate that the strength or peak-to-peak values of the multi-polar acoustic signals may be used to help identify the material of interest. Since the reference is made of a known material with a known absorption property and the strength or peak-to-peak values of the multi-polar acoustic signals depend on the relative absorption properties of the materials, the absorption property of the material of interest can be deduced.

Those skilled in the art will appreciate that in some embodiments the thermoacoustic data may need to be corrected. For example, the shape of the boundary may be deformed. In this embodiment, the shape and/or angle of the boundary may be estimated using the ultrasound imaging system and known image processing techniques.

In this embodiment, received signals at the thermoacoustic transducer array may be expressed using equation 12:

pₛ(t)=∫ₛ p(r, t)dS (12)

where S is the surface area of the thermoacoustic transducer array. As will be appreciated, the properties of the thermoacoustic transducer array and its positioning relative to the region of interest change the characteristics of the thermoacoustic data. The multi-polar acoustic signals received by the thermoacoustic transducer array are affected by various factors that are not related to signal generation by rather associated with signal propagation. These factors depend on transducer spatial sensitivity, relative positioning between the thermoacoustic transducer array and the boundary between the material of interest and the reference, and the relative shape of the reference with respect to the thermoacoustic transducer array surface. Even for the same region of interest and the same thermoacoustic transducer array, changing the position and angle of the thermoacoustic transducer array during thermoacoustic data acquisition results in different measurements.

In this embodiment, a compensation factor is calculated based on information and measurements provided by the operator or estimated using acquired ultrasound image data. The compensation factor may be a single factor or multiple factors, where each factor is calculated information such as size and shape of the reference and the angle between the ultrasound transducer array and the boundary. In one embodiment, the compensation factors are calculated based on theoretical methods such as by using acoustic propagation and ultrasound transducer properties. In another embodiment, the compensation factors may be obtained from phantom and clinical studies. In yet another embodiment, both theoretical and experimental methods may be used.

When the thermoacoustic data is adjusted with the compensator factor, the thermoacoustic signal strength, Sₜ, in equation 11 is replaced by the adjusted thermoacoustic signal strength, Sₜ:

$${k}_{MOI} = {Γ}_{MOI} {ɛ}_{r , MOI}^{″} = \frac{{\underline{s}}_{l}}{{{ωɛ}_{0} ( {E}_{0} {e}^{- {n}_{ref} {d}_{ref}} )}^{2}} + {Γ}_{ref} {ɛ}_{0} {ɛ}_{r , ref}^{″} \qquad ( 13 )$$

In this, when the material of interest is large enough to ignore the partial volume effect, only the angle based adjustment is required. When a tangent vector of the material of interest at the boundary and the centerline of the one or more transducer arrays 34 is not perpendicular, signal adjustment is made to the acquired measurement. This adjustment is expressed as:

[symbol]

=SₗC(θ)

where C is an angle based adjustment factor, θ is the angle between the tangent vector of the material of interest at the boundary and the centerline of the one or more transducer arrays 34.

Although in embodiments described above, Tables 1 and 2 are described as being used to identify the material of interest, those skilled in the art will appreciate that additional or alternative tables may be used. For example, the material may be identified using a lookup table comprising a function of the multi-polar acoustic signals, a forward power of the energy signal and the VSWR of the energy signal. Table 3 shows an exemplary lookup table:

TABLE 3
Lookup Table to Identify the Material of Interest

| | | |
|---|---|---|
| Measurements | Material 1 | Material 2 |

| | | |
|---|---|---|
| Quantitative | Thermoacoustic | Material independent |
| | | characteristic TA signal |
| | | Corrected TA signal at the |
| | | reference to material |
| | | boundary |
| | | Characteristic TA property |
| | | (Grüneisen x Conductivity) |
| | Power measurements | Forward |
| | (no reference) | |
| | | Transmitted |
| | | VSWR |
| | Power measurements | Forward |
| | (with reference) | |
| | | Transmitted |
| | | VSWR |
| | Combined metrics | Ratio between TA signals |
| | | Function of ratio between TA |
| | | signals and TA properties |
| | | Function of TA signal, |
| | | properties, and power |
| | | measurements |
| | Ultrasound | Speed of sound |
| Semi- | Ultrasound | Scattering |
| quantitative | | |
| Other | Color | |
| characteristics | | |
| | Transparency | |
| | Texture | |
| | Material state | |
| | Odor | |

Semi-quantitative measurements may be used to help identify the material of interest. For example, for a material with an unknown condition, each quantitative property may be compared against conditions listed in Table 3. An exemplary comparison metric is shown in equation 15:

Qᵢ=Σⱼ₌₁^{N}(p^{j}ᵢ−p^{j}_{unknown})w^{j} (15)

where N is the total number of quantitative property being included in the comparison, i denotes the i^{th} condition in the lookup table, j denotes j^{th} material property, p^{j}ᵢ denotes the j^{th} property of material with i^{th} condition p^{j}_{unknown} denotes the j^{th} property of the material with unknown condition, w^{j} denotes a weighting factor for j^{th} property. Material with the lowest Qᵢ will be considered as the identified condition of the material. As will be appreciated, only part of the materials properties may be used to determine the material condition.

In some embodiments, the identity of the material will not be directly selected from the lookup table, but the likelihood of its identity will be suggested based on the acquired metrics. For example, a material may have 60% likelihood to be material 1 and 40% likelihood to be material 2.

In some embodiments, the identity of material will be proposed as a weighted combination of multiple materials, based on the acquired metrics.

In some embodiments, the parameter or the condition of the material of interest will be proposed as a weighted combination of multiple materials, based on the acquired metrics.

In some embodiments, various measurements from thermoacoustic data can be used to construct a parameter, which may be tabulated and used to help identify the material. For example, thermoacoustic data having no object related signals may be obtained at the beginning of the measurements. For example, thermoacoustic data may be obtained with the coupling medium (e.g., ultrasound gel) being present, thermoacoustic data may be obtained and may comprise signals generated from internal components or transducer components, the boundary between the reference and the RF applicator, the internal components of the RF applicator, or other parts of the RF applicator. Thermoacoustic data from such sources are independent of the material and can be used to compensate or normalize the thermoacoustic signal from the material. A simple ratio between thermoacoustic data obtained at the reference to the boundary and thermoacoustic data having no object related signals may be used as a metric. As will be appreciated, various combinations of thermoacoustic data having no object related signals may be obtained.

Those skilled in the art will appreciate that in embodiments, the boundary between the reference and the material of interest may be automatically defined using algorithms based on ultrasound segmentation or thermoacoustic data analysis. As will be appreciated, both operator-defined and automatic methods may be combined.

Although the reference is described as being in the form of a pad made of a known material and is placed on top of the material of interest, those skilled in the art will appreciate that alternatives are available. For example, in another embodiment the reference may be in the form of a container or petri dish configured to hold or contain the material of interest.

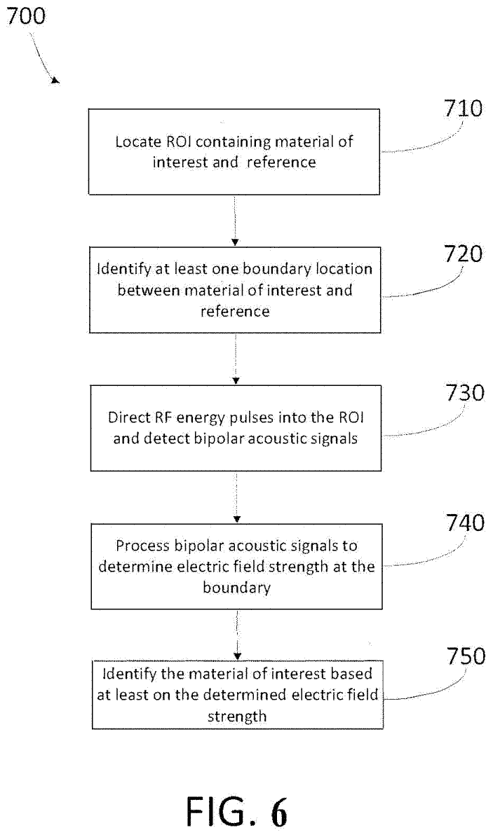

Turning now to FIG. 6, another embodiment of a method for identifying a material of interest is shown and is generally identified by reference numeral 700. Method 700 is generally identical to that of method 500 with the following exceptions. During method 700, rather than the region of interest being in vitro, in method 700 region of interest is in vivo. As such, initially during the method, a region of interest ROI to be imaged that contains a material of interest and a reference separated by at least one boundary is located within a subject's body (step 510). In this embodiment, the region of interest ROI is located using the ultrasound imaging system 24. Specifically, ultrasound image data obtained by the ultrasound imaging system 24 is communicated to the computing device 22. The ultrasound image data is processed by the computing device 22 and a reconstructed ultrasound image is presented on the display device. The operator moves the ultrasound transducer 28 until the region of interest is located. When locating the region of interest, the computing device 22 overlays information associated with the angle of the centerline of the one or more transducer arrays 30 of the ultrasound transducer 28 overtop of the reconstructed ultrasound image on the display device. The information is used to provide feedback to the operator to ensure the axial axis of the ultrasound transducer 28 is generally perpendicular to a boundary between the object of interest and the reference.

Once the region of interest ROI has been located, steps 720, 730, 740 and 750 of method 700 are generally identical to that of steps 520, 530, 540 and 550 of method 500, respectively.

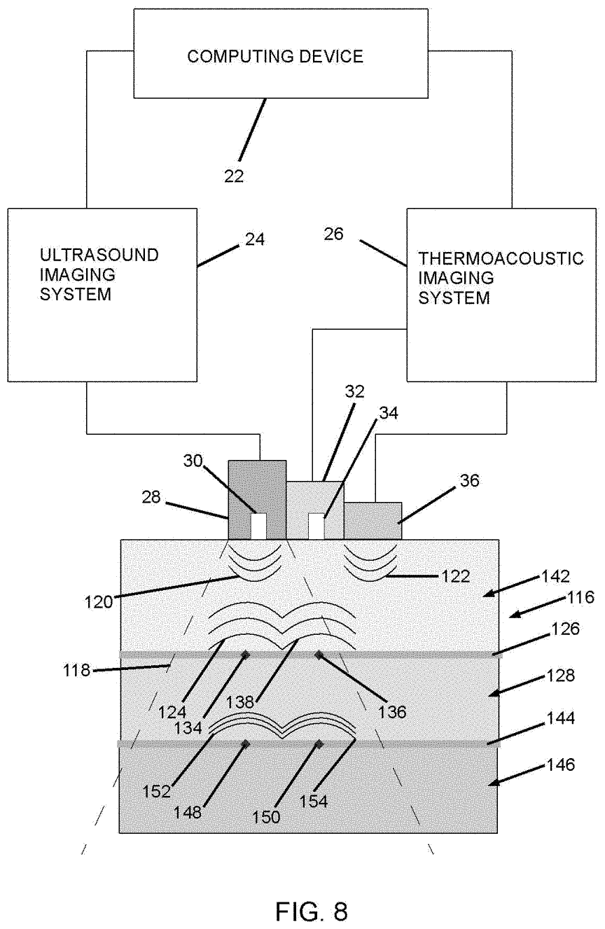

Although in embodiments a single boundary, a single material of interest and a single reference are imaged, those skilled in the art will appreciate that alternatives are available. An example is shown in FIG. 8. In this embodiment, a user utilizes the computing device 22 to operate the ultrasound imaging system 24. The ultrasound imaging system 24 sends a signal to the one or more ultrasound transducer arrays 30, which sends sound waves 120 into reference 142 which is in the form of a pad made of a known material and is positioned within the region of interest 116. Within the region of interest 116, there is a first boundary 126 at first boundary locations 134 and 136, a first material of interest 128, a second material of interest 146, and a second boundary 144 at second boundary locations 148 and 150. The ultrasound transducer arrays 30 receive reflected sound waves to generate a B-mode image via the ultrasound imaging system 24. The extent of the B-mode image is conical in shape and is shown with B-mode image limits 118. The B-mode image gives the physical location of boundary 126, enabling the computing device 22 to correlate obtained data from the thermoacoustic transducer array 34 and RF emitter 36. Once position coordinates are known, the ultrasound imaging system 24 is turned off to eliminate potential interference with the thermoacoustic imaging system 26.

The thermoacoustic imaging system then initiates the RF emitter 36 to send RF energy pulses 122 into reference 142. The RF energy 122 pulses are absorbed in the region of interest 116. The difference between RF energy absorbed in the reference 142 and the first material of interest 128 creates multi-polar acoustic signals 124 and 138 emanating from boundary locations 134 and 136. In addition, the difference between RF energy absorbed in the first material of interest 128 and the second material of interest 146 creates multi-polar acoustic signals 152 and 154 emanating from boundary locations 148 and 150. The known parameters of the reference 142 can then be used in conjunction with thermoacoustic data from the multi-polar acoustic signals generated at boundary locations 134 and 136 to determine a parameter of the first material of interest 128. Additionally, properties or parameters of the first material of interest 128 can be used in conjunction with thermoacoustic data from the multi-polar acoustic signals generated at boundary locations 148 and 150 to determine a parameter of the second material of interest 146.

Although in embodiments described above the one or more ultrasound transducer arrays are described as being disconnectable from the ultrasound imaging system and reconnectable to the thermoacoustic imaging system, those skilled in the art will appreciate that alternatives are available. For example, the one or more ultrasound transducer arrays may be connected to a hub which itself is connected to the ultrasound imaging system and the thermoacoustic imaging system. In this embodiment, the hub may be controlled by the computing device or by other input to switch operation between the ultrasound imaging system and the thermoacoustic imaging system and vice versa.

Although in embodiments described above the thermoacoustic data is described as being in the form of multi-polar acoustic signals, those skilled in the art will appreciate that the thermoacoustic data may be in the form of other multipolar signals.

Although in embodiments described above a metric used to estimate the signal strength at the boundary is the difference between two or more peaks of a multipolar signal, those skilled in the art will appreciate that the metric may be a simple peak (maximum), a p-norm, area under the multipolar signal, etc.

As will be appreciated, embodiments of image processing described above can be performed on ultrasound and thermoacoustic images in real-time or off-line using images stored in memory.

Although the thermoacoustic imaging system is described as comprising an RF applicator configured to generate short pulses of RF electromagnetic radiation, those skilled in the art will appreciate that in other embodiments the thermoacoustic imaging system may comprise a visible light source or an infrared radiation source with a wavelength between 400 nm and 10 μm and a pulse duration between 10 picoseconds and 10 microseconds.

Although in embodiments described above the thermoacoustic imaging system and the ultrasound imaging system are described as using one or more ultrasound transducer arrays, those skilled in the art will appreciate that the alternatives are available. For example, a single transducer element, an ultrasound transducer array having a linear or curved one-dimensional array, or a two-dimensional ultrasound transducer array may be used.

Although in embodiments described above, thermoacoustic data is obtained of a single region of interest, those skilled in the art will appreciate that multiple regions of interest may be analyzed and combined.

Those skilled in the art will appreciate that the above-described ultrasound image data and thermoacoustic data may be one-dimensional, two-dimensional or three-dimensional. In embodiments, the ultrasound image data may be in a different dimension than the thermoacoustic data. For example, ultrasound image data may be two-dimensional and the thermoacoustic data may be one-dimensional. Further, different fields of view may be used.

In another embodiment, different types or models of transducer arrays may be used with the thermoacoustic and ultrasound imaging systems. In this embodiment, a transform may be used to map a thermoacoustic absorption data to the ultrasound image. In another embodiment, in the event that knowledge of transducer array geometry is not readily available, the thermoacoustic absorption data may be mapped to the ultrasound image using a phantom with reference points. In this embodiment, a transform may be used to map known phantom reference points from the thermoacoustic absorption data to the phantom reference points on the ultrasound image.

Although the ultrasound imaging system is described as using B-mode ultrasound imaging techniques, other techniques may be used such as for example power Doppler images, continuous wave Doppler images, strain imaging, etc.

Although in embodiments described above thermoacoustic data is obtained of the region of interest, those skilled in the art will appreciate that thermoacoustic data may be obtained for an area larger than the region of interest.

As described above, the programmed computing device executes computer program code stored on at least one computer readable medium. The computer readable medium may be memory devices or transmitting devices, thereby making a computer program product or article of manufacture according to the invention. As such, functionality may be imparted on a physical device as a computer program existent as instructions on any computer-readable medium such as on any memory device or in any transmitting device, that are to be executed by a processor.

Examples of memory devices include, hard disk drives, diskettes, optical disks, magnetic tape, semiconductor memories such as FLASH, RAM, ROM, PROMS, and the like. Examples of networks include, but are not limited to, the Internet, intranets, telephone/modem-based network communication, hard-wired/cabled communication network, cellular communication, radio wave communication, satellite communication, and other stationary or mobile network systems/communication links.

A machine embodying the invention may additionally or alternatively include one or more processing systems including, for example, computer processing unit (CPU) or processor, memory/storage devices, communication links, communication/transmitting devices, servers, I/O devices, or any subcomponents or individual parts of one or more processing systems, including software, firmware, hardware, or any combination or subcombination thereof.

Using the description provided herein, embodiments described above may be implemented as a machine, process, or article of manufacture by using standard programming and/or engineering techniques to produce programming software, firmware, hardware or any combination thereof.

Using the description provided herein, those skilled in the art will be readily able to combine software created as described with appropriate or special purpose computer hardware to create a computer system and/or computer subcomponents embodying the invention, and to create a computer system and/or computer subcomponents for carrying out at least some steps of the methods described herein.

Those skilled in the art will appreciate that in embodiments the computing device 22 may be programmed to adjust parameters of the RF applicator.

Those skilled in the art will appreciate that the above-described method may be performed on a phantom designed to mimic an area of interest. In this embodiment, the RF applicator may be adjusted to maximize the peak-to-peak amplitudes of the multi-polar acoustic signals prior to imaging a material. Further, the method may be performed on numerous phantoms of various properties to mimic different materials.

Although embodiments have been described above with reference to the accompanying drawings, those of skill in the art will appreciate that variations and modifications may be made without departing from the scope thereof as defined by the appended claims.

What is claimed is:

1. A method for identifying a material of interest, the method comprising:
directing, using a radio frequency (RF) applicator, one or more RF energy pulses into a region of interest, the region of interest comprising the material of interest and at least one reference that are separated by at least one boundary;
detecting, using an acoustic receiver, at least one multi-polar acoustic signal generated in the region of interest in response to the RF energy pulses;
processing the at least one multi-polar acoustic signal to determine an electric field strength at the boundary; and
identifying the material of interest based at least on the determined electric field strength.

2. The method of claim 1 wherein the identifying comprises calculating a parameter as a product of a Grüneisen Parameter of the material of interest and a conductivity of the material of interest.

3. The method of claim 2 wherein the identifying comprises looking up the calculated parameter in a lookup table.

4. The method of claim 1, wherein the electric field strength is determined based on an input power of the RF applicator and an attenuation coefficient of the reference.

5. The method of claim 1, wherein the electric field strength is determined based on an estimated thickness of the reference and an attenuation coefficient of the reference.

6. The method of claim 1 wherein each multi-polar acoustic signal corresponds to a separate boundary location.

7. The method of claim 1, wherein detecting the at least one multi-polar acoustic signal is achieved using a thermoacoustic imaging system.

8. The method of claim 7, further comprising:
directing, using an ultrasound system, sound waves into the region of interest;
detecting, using an ultrasonic transducer of the ultrasound system, echoes generated in the region of interest in response to the sound waves; and
processing ultrasound data associated with the echoes to generate one or more or more ultrasound images.

9. The method of claim 1, wherein identifying the material of interest is further based on at least one physical characteristic of the material of interest.

10. The method of claim 9, wherein the at least one physical characteristic is at least one of color, transparency, odor, texture and material state.

11. The method of claim 1 wherein the material of interest is tissue within a human body and the reference is lean tissue within the human body.

12. A system for identifying a material of interest, the system comprising:
a thermoacoustic imaging system comprising a radio frequency (RF) applicator configured to emit RF energy pulses into the region of interest comprising a material of interest and a reference separated by at least one boundary and an acoustic receiver configured to receive at least one multi-polar acoustic signal generated in the region of interest in response to the RF energy pulses; and
one or more processors configured to:
process multi-polar acoustic signals received by the acoustic receiver to determine an electric field strength at the boundary; and
identify the material of interest based at least on the determined electric field strength.

13. The system of claim 12 wherein during the identifying the one or more processors are configured to:
calculate a parameter as a product of a Grüneisen Parameter of the material of interest and a conductivity of the material of interest.

14. The system of claim 12 wherein during the identifying the one or more processors are configured to:
look up the calculated parameter in a lookup table.

15. The system of claim 12 wherein the electric field strength is determined based on an input power of the RF applicator and an attenuation coefficient of the reference.

16. The system of claim 12 wherein the electric field strength is determined based on an estimated thickness of the reference and an attenuation coefficient of the reference.

17. The system of claim 12 wherein each multi-polar acoustic signal corresponds to a separate boundary location.

18. The system of claim 12 wherein the material of interest is tissue within a human body and the reference is lean tissue within the human body.

19. The system of claim 12 wherein the reference is made of a known material and is placed adjacent to the material of interest.

20. The system of claim 12, wherein the reference is one of a container and a pad.

21. A non-transitory computer readable medium having stored thereon computer program code executable by one or more processors to:
process at least one multi-polar acoustic signal generated in a region of interest comprising a material of interest and at least one reference that are separated by at least one boundary to determine an electric field strength at the boundary; and
identify the material of interest based at least on the determined electric field strength

22. The non-transitory computer readable medium of claim 21 wherein the computer program code is executable by the one or more processors to:
calculating a parameter as a product of a Gr\ddot{n}eisen Parameter of the material of interest and a conductivity of the material of interest.

23. The non-transitory computer readable medium of claim 21 wherein the computer program code is executable by the one or more processors to:
identify the material of interest by looking up the calculated parameter in a lookup table.
